(19) United States
(12) Patent Application Publication
(10) Pub. No.: US 2025/0186687 A1
(43) Pub. Date: Jun. 12, 2025

(54) VENOCLYSIS DEVICE

(71) Applicant: Armando Jorge Caso Y Macmaster, Ciudad de Mexico (MX)

(72) Inventor: Armando Jorge Caso Y Macmaster, Ciudad de Mexico (MX)

(21) Appl. No.: 18/535,617

(22) Filed: Dec. 11, 2023

(51) Int. Cl. A61M 5/155; A61M 5/142

(52) U.S. Cl. CPC A61M 5/155; A61M 5/14244; A61M 2209/088

(57) ABSTRACT

This invention hereby is associated with the field of medicine and the pharmaceutical industry, such as venoclysis equipment, syringes, among others. More specifically, it is associated with the manufacturing of a venoclysis device, which, due to a chemical reaction, allows for the generation of sufficient pressure in order to establish a permeable pathway between a solution container and the venous bloodstream characterized by consisting of a casing in which there are two containers, one of them consisting of liquid or serum, the other one consisting of a mixture of salts and an additional container with an aqueous solution of a weak or organic acid.

### FIELD OF THE INVENTION

The following invention is related to the field of medicine and the pharmaceutical industry, such as venoclysis equipment, syringes, among others. More particularly, it is related to the manufacturing of a venoclysis device, which, thanks to a chemical reaction, allows generating sufficient pressure to establish a permeable pathway between a solution container and the venous bloodstream.

### BACKGROUND OF THE INVENTION

The medical term venoclysis or peripheral venous catheterization means the introduction of fluid into a vein lumen and it refers to establishing a permeable pathway between a solution container and the venous bloodstream. This is achieved by making a percutaneous puncture into the venous lumen and inserting a catheter into the vein. Therefore, when the solution flows into the bloodstream, a permeable venous route is established.

The vein puncture is carried out for three main reasons: to obtain blood, to supply some substance to the bloodstream or to introduce some instrument for diagnosis or treatment.

Venoclysis to obtain blood. Obtaining blood samples is necessary to carry out laboratory studies such as hematology, blood chemistry, special tests and blood cultures. In this case, tools such as pericranial catheters (commonly called butterfly needles or scalp vein set) or vacutainers are utilized, whereby both are designed for brief use. Once the sample is taken, these devices are removed.

Venoclysis for supplying purposes. The vein puncture can be carried out in order to supply something to the body, such as hydrating solutions, medicines, nutrients, contrast media to carry out imaging studies or blood transfusions. In this case, catheters designed to remain in the vein for several hours or days are utilized, such as the jelco catheter, which has the advantage that after the puncture a plastic tube is introduced, allowing the area to be mobilized without the risk of tissue damage.

Venoclysis for monitoring purposes. Sometimes venoclysis is performed to introduce devices into the bloodstream in order to make a diagnosis, such as cardiac catheterization or central venous pressure monitoring.

The principles that this technique is based on are the following: the vein pressure is greater than atmospheric pressure, so if a solution is injected into a vein, it must be done at a higher pressure to overcome that of the vein. When the pressure of the fluid to be introduced is lower than the blood pressure, the puncturing of the vein walls causes bleeding and it can cause bruising.

The pressure of the liquid to be introduced into the bloodstream is normally generated by the liquid column that is above the level of the patient's body, whereby said pressure overcomes the blood pressure and it allows passage of the solution through the vein, based on Bernoulli's principle which tells us that, within a constant flow of energy, when the fluid flows through a region of lower pressure, it accelerates and vice versa. Thus, this makes it necessary for a tripod to be used in order to favor said force.

In the technique's condition, it is known that, when applying an aqueous solution of citric acid, to sodium bicarbonate, it results in the production of gaseous carbon dioxide which generates a pressure that can, for example, inflate a balloon.

Currently there are some teams already seeking to optimize the process of transporting the solution during venoclysis. Such is the case of PCT WO 2020016110 A1, where Shirafi Mahmoudreza is presenting a device and equipment to monitor multiple venoclysis sets, through a method to administer a medication that includes measuring the dosing rate of a first intravenous bag connected to a first infusion pump, establishing a pumping rate of the first infusion pump, comparing the pumping rate of the first infusion pump with the measured dosing rate of the first intravenous bag, and adjusting the pumping rate of the first infusion pump to the measured dosing rate of the first intravenous bag when the pumping rate of the first infusion pump is different from the measured dosing rate of the first intravenous bag. This PCT exhibits the problem which requires that all solutions have to be at a certain height to achieve the process, as well as the space requirement that is necessary for its use.

Also, in the CA 3058462 A1 patent in which Booth David E, Despa Mircea, et al, are presenting an infusion device to infuse a medical fluid to a patient which consists of a casing configured to receive a container filled with the medical fluid, at least partially. Furthermore, the infusion device has a driving mechanism associated with the casing configured to deliver the medical fluid from the container to the patient in a dosing procedure. The delivery device further has a module configured to detect at least one property of the dosing procedure and a property of the medical fluid. The module has at least one dose detection sensor configured to detect the initiation, progression, and completion of the dosing procedure based on the position of a stopper within the container. Furthermore, the module has at least one temperature sensor configured to measure the temperature of the medical fluid within the container based on the temperature of the container. Said patent poses the problem of requiring a power source to operate it, either with batteries or by pre-charging it, which is necessary for its operation.

Patent number CN112843377A involves an invention relating to an infusion holder, in particular a multifunctional infusion holder with an adjustable angle. The purpose of this invention is to provide a multifunctional infusion holder that can adjust the position and angle of the infusion holder, guide the infusion tube without easily shaking it, and facilitate the movement and placement of the infusion holder.

A multifunctional infusion holder with adjustable angle includes: a base and an adjustable assembly, the top of the base is provided with the adjustable assembly. The invention achieves the following: when infusing the patient, the infusion tube can be guided through the guide ring. It can effectively prevent the infusion tube from moving during the patient's infusion, which would have a disastrous impact when introducing the liquid.

The problem posed by the technique's condition is that it is based on the use of a tripod or support to begin the venoclysis process and the use of electrical energy is also necessary for its operation.

On the other hand, it necessarily requires a means, if not the tripod, any other support, to keep the bag or container in place in order to introduce the liquid into the patient's bloodstream, at a height differential with respect to said patient.

### SUMMARY OF THE INVENTION

The main objective of this invention is to create a venoclysis equipment that can be used without having to use an external support such as a tripod for its operation.

Another objective is to achieve the above through a simple mechanism, that is maintenance-free and easy to use by the operator in view that just breaking a small bag with a solution is enough to start gas production.

Yet another objective is to ensure that when said venoclysis device is in under its closed modality, it increases the volume of one of the bags, thereby pressing the bag with the solution to be transfused, and thus transferring said solution.

And all those advantages and objectives that will become apparent after reading this description hereby, which include but are not limited to the images that are an integral part of this document.

In general, this invention hereby relates to venoclysis devices, and it is characterized by being able to accommodate two valves.

Inside the valves, when joined together, they form a rigid container or receptacle, where 2 bags are contained, one of them contains the substances that upon reaction will result in the propellant and the other the liquid to be transfused.

The substances that will result in the propellant are two, an organic acid and a carbonated salt. The organic acid will be in an aqueous solution and it may be acetic acid or citric acid, and the salt is chosen from sodium bicarbonate and/or calcium carbonate. The aqueous solution is contained in a small, frangible bag and the salt is free or dispersed within the bag of the propellant generating elements. To generate the propellant, in this case carbon dioxide, the frangible bag must be broken to bring its contents into contact with the salt.

The other bag, the one that contains the liquid to be introduced into the bloodstream and which has a connector coming out of it with a regulating lock to be able to standardize the liquid flow through the vein and at its most distant end it has a drip chamber and a catheter. The connector is hydraulically attached to the bag, and it crosses the bivalve receptable outwards.

Then, these two bags, the one with the propellant generating elements and the one with the liquid to be transfused, can be one on top of the other or one next to the other.

To hold the two valves together, one or two hook and loop fasteners (Velcro™) may be used, or by any other suitable means, such as so-called snaps, or padlock slot, or any other attachment means.

By producing the carbon dioxide in one of the bags, it is inflated and pressed onto the bag with the liquid to be transfused thus propelling it and making it flow through the catheter.

### BRIEF DESCRIPTION OF THE DRAWINGS

FIG. 1 shows a zoomed-out image of all the components of the venoclysis device.

FIG. 2 illustrates FIG. 1, showing how the serum bag and the gas-generating bag are positioned to produce the pressure necessary to infuse said serum.

FIG. 3 shows a conventional perspective image of the assembled device.

### DETAILED DESCRIPTION OF THE INVENTION

To elaborate on this chapter, the modalities illustrated in the attached images will be used.

The venoclysis device allows for the solutions to be transferred in a different manner, generating the necessary pressure and allowing its placement near the user, without having to use a tripod, which allows for greater portability and a larger area for its use is not needed.

The interaction of the necessary amount of sodium bicarbonate or calcium carbonate, with the aqueous solution of citric acid (or acetic acid), will generate the carbon dioxide that will create the necessary pressure to drive the transfusable solution through the veins.

The venoclysis device conformation shows a degree of complexity due to the configuration of both containers, whereby one is for the components that will generate the pressure (propellant) in the mechanism and the other one is where the transfusable solution is contained.

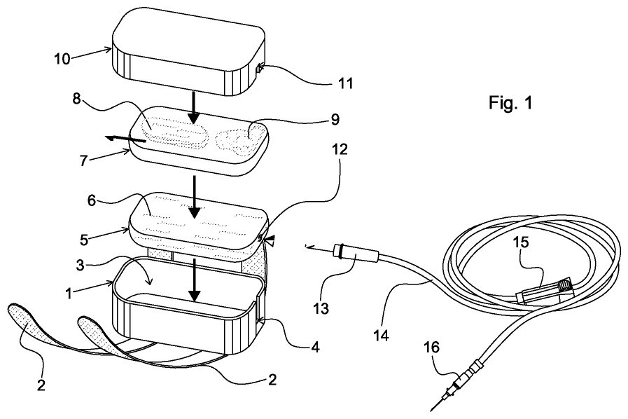

FIG. 1 shows a conventional perspective breakdown of the venoclysis device 1, whereby said device 1 has, in its upper part, a structure of valve 10, which has such a space that it allows for the placement of a first container 7, likewise it has a concave space 11. Container 7 holds 1 bag 8, which contains the organic acid solution, whereby said container 8, once broken, releases the acid solution letting it contact mixture 9 and generate the pressure, which is dispersed in the same container 7. Under this modality, the lower part of the device consists of bag 5 which has the venoclysis liquid and at its right end it has a hole through which a connector attached to drip chamber 13 will be inserted, and once the necessary pressure has been generated, it will push the liquid through flexible tube 14, regulator lock 15 is located at the distal end of flexible tube 14 and next to it is catheter 16.

Valve 1 has slot 4 that crosses the side wall, which coincides with opening 12 of solution bag 6. At the bottom of the base, a pair of fastener systems are located, from the device to the patient.

Other modalities of the invention will involve the attachment means of the valves to form the casing. One preferred modality comprises the means of attaching the valves by using hook and loop fasteners (Velcro™). Other preferred attachment means are so-called snaps, but bolt padlock attachment means may also be used. There is also the edge tab attachment means.

Although the combination of two simultaneous attachment means is not mentioned in any image, a device with two attachment means is also possible. For example, a preferred modality consists of two valves attached by snaps and hook and loop fasteners means. The straps would be used to attach the device to the patient.

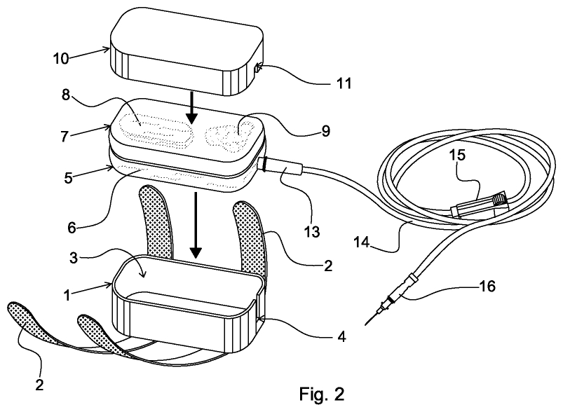

FIG. 2 illustrates FIG. 1, showing how the serum bag and the gas-generating bag are positioned to produce the pressure necessary to infuse said serum. This is the preferred modality, but another preferred modality would be for the bags to be placed edgewise. The elements of this image are already indicated in the previous image and the only thing that changes is the arrangement of the bags in the rigid placement.

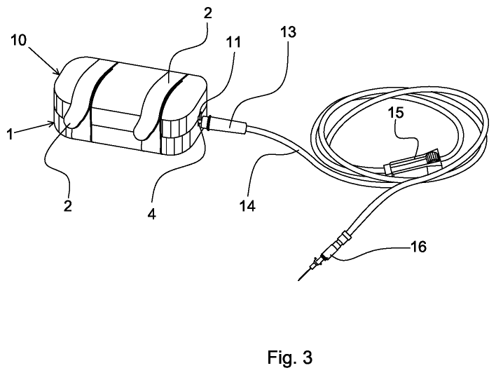

FIG. 3 shows a conventional perspective image of the assembled device, with base 1 and lid 10 completely closed and forming a cavity, which is formed due to the parts 11 and 4, in the central part of the lateral face of said prism. This cavity is located at the same height as orifice 12 of solution bag 5. Clamping device 2 is gripping the entire device.

Another modality of this invention hereby involves the same container 7 in order to place a bag, which contains an aqueous solution of organic acid, for example, acetic acid or citric acid and, upon breaking, mixture 9 comes into contact thus generating pressure, which is dispersed in the same container 7, whereby said pressure-generating mixture can be composed of sodium bicarbonate and calcium carbonate.

Yet another modality of this invention hereby involves the same container 7 in order to place a bag, which contains the organic acid solution and, upon breaking, mixture 9 comes into contact thus generating pressure, which is dispersed in the same container 7, whereby said pressure-generating mixture can be composed of acetic acid and citric acid.

In addition, another modality of this invention hereby involves the same container 7 in order to place a bag, which contains the aqueous solution of the organic acid and, upon breaking, mixture 9 comes into contact thus generating pressure, which is dispersed in the same container 7, whereby said pressure-generating mixture can be composed of sodium bicarbonate and citric acid.

Likewise, there is another possible modality of the invention, involving the same container 7 in order to place another container with aqueous solution of organic acid and the mixture of bicarbonate or carbonate is covered with oil, whereby this is in order to gradually control the production of gas.

Additionally, another preferred modality is to form a matrix of an appropriate water-soluble polymer, wherein the acids and organic salts are encapsulated, and as the polymer dissolves, it allows the water to come into contact with the bicarbonate and/or carbonate mixture and carry out the reaction.

A preferred modality comprises a carbon dioxide forming reaction retarding gel.

Conformation of the receptacle that will hold the containers or bags with the water and the reagents and the solution to be transfused, may vary significantly. This receptacle under the preferred modality has the shape of a parallelepiped, but it may also have the shape of a kidney, or an ellipsoid, among others.

The final presentation of the device also has different modalities, because having solved the self-propelling mechanism of the device, there are other accessories that depend on the technique's condition, but which can also be used and sometimes must be used by this invention hereby.

These accessories include the flexible tube in order to conduct the fluid to be transfused, and obviously the cannula to be introduced into the required vein or artery. The drip chamber and the flow control valve.

A preferred modality is to place these accessories inside the casing along with the bags, but another preferred modality is also to place said accessories in a separate bag. This bag and its contents would have to be sterile.

The invention has been thoroughly described so that a person with average skills on the subject matter can reproduce and obtain the results mentioned in this invention hereby. However, any person skilled in the technique's field within the scope of this invention hereby may be able to make modifications not described herein; however, if the subject matter required falls under the following claims in order to apply these modifications in a given product or in the manufacturing process thereof, said structures should be confined within the scope of the invention.

1. A venoclysis device comprising:
a closed placement unit (casing) where there are two containers (bags), one of them containing the liquid or serum of any type, to be perfused, the other one containing a mixture of salts (sodium bicarbonate or calcium carbonate), and an additional container with an aqueous solution of an organic acid.

2. The venoclysis device, as claimed in the preceding claim, which is characterized in that the casing has a slightly larger volume with regards to the containers inside.

3. The venoclysis device, as claimed under claim 1, which is characterized in that the salt mixture consists of sodium bicarbonate and aqueous citric acid solution.

4. The venoclysis device, as claimed under claim 1, which is characterized in that the gas generation mixture includes a retardant, to control the reaction rate from the inflow rate of the liquid to be perfused, when entering the patient's vein.

5. The venoclysis device, as claimed under claim 2, is characterized in that once the casing is assembled, a closing device is used to secure the contents.

6. The venoclysis device, as claimed in claim 4, is characterized in that the casing is composed of two halves, an upper and a lower one, with an attachment means to keep them closed and without opening when the pressure is increased by the gas generation in the bag where the pouch of aqueous solution of organic acid and the mixture of salts is located.

7. The venoclysis device, as claimed under claim 4, is characterized in that the attachment means consists of straps with their ends comprising a hook and loop (Velcro™) attachment means, and each transversely embraces the two parts of the casing.

8. The venoclysis device, as claimed under claim 7, is characterized in that it comprises two attachment means, one is in order to keep the two-valve casing closed and the other one is in order to fasten the device to one of the patient's extremities.

9. The venoclysis device, as claimed under claim 1, is characterized in that the self-propelling device is distributed accompanied by a bag in which the required accessories are arranged in a venoclysis equipment.
